(19) United States
(12) Plant Patent Application Publication
(10) Pub. No.: US 2012/0255088 P1
(43) Pub. Date: Oct. 4, 2012

(54) Gaura plant named 'KLEGL10716'

(50) ;

(75) Inventors: Nils Klemm, Stuttgart (DE); Martin Glawe, Stuttgart (DE)

(73) Assignee: KLEMM+SOHN GMBH & CO., KG, Stuttgart (DE)

(21) Appl. No.: 13/065,807

(22) Filed: Mar. 30, 2011

(51) Int. Cl. A01H 5/00

(52) U.S. Cl. PLT/432

(57) ABSTRACT

A new variety of Gaura plant named ‘KLEGL10716’, particularly characterized by an upright and bushy habit, good outdoor garden performance, and white flowers that last 3 to 4 days on the plant.

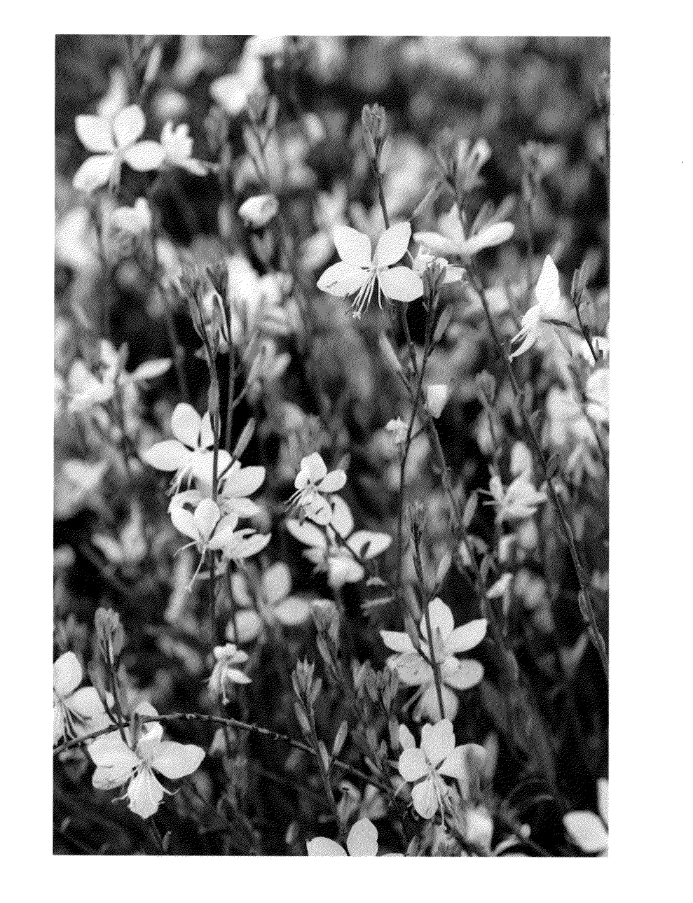

### GENUS AND SPECIES

Gaura lindheimeri

### VARIETY DENOMINATION

‘KLEGL10716’

### BACKGROUND OF THE NEW PLANT

The present invention comprises a new and distinct variety of Gaura plant, botanically known as Gaura lindheimeri, and hereinafter referred to by the variety name ‘KLEGL10716’. The new Gaura variety was discovered as a seedling in Stuttgart, Germany. A single plant selection was subsequently chosen for further evaluation and asexual propagation.

The new Gaura variety ‘KLEGL10716’ was first propagated via vegetative cuttings in Stuttgart, Germany in April, 2009 and has been asexually reproduced repeatedly by vegetative cuttings in Stuttgart, Germany for approximately 3 to 4 generations. The new Gaura variety ‘KLEGL10716’ has been found to retain its distinctive characteristics through successive asexual propagations via vegetative cuttings.

Plant Breeder's Rights for this variety have not been applied for. ‘KLEGL10716’ has not been made publicly available or sold more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. White flowers;

2. Good outdoor performance;

3. Upright, bushy habit; and

4. Flowers that last 3 to 4 days on the plant.

### DESCRIPTION OF THE PHOTOGRAPH

The new Gaura variety ‘KLEGL10716’ is illustrated by the accompanying photograph which shows blooms, buds, and foliage of the plant. The colors shown are as true as can be reasonably obtained by conventional photographic procedures. The photograph is of a 12-week old plant taken in Stuttgart, Germany and grown from vegetative cuttings in a 12 centimeter plastic container in a glass greenhouse in Stuttgart, Germany under conditions which approximate those generally used in commercial practice.

### DESCRIPTION OF THE NEW VARIETY

The following detailed descriptions set forth the distinctive characteristics of the new Gaura variety ‘KLEGL10716’. The data which define these characteristics were collected from asexual reproductions carried out in Stuttgart, Germany from January through May of 2010. The plant history was taken on 3-month old plants that were grown in 12 centimeter containers in a glass greenhouse in Stuttgart, Germany. The plants were pinched once 10 days after planting. The color readings were determined under natural light. Color references are to the RHS Colour Chart of The Royal Horticultural Society of London (R.H.S.), Fifth Edition (2007).

### DETAILED BOTANICAL DESCRIPTION OF THE NEW PLANT

Classification:
   Family name.—Gaura.
   Botanical name.—Gaura lindheimeri.
   Common name.—Gaura Belleza White.
Plant:
   Habit.—Upright, bushy.
   Height.—40 cm (when grown in a container).
   Spread.—20 to 25 cm (when grown in a container).
   Outdoor or garden performance.—Good, upright, 40 to 60 cm.
Propagation:
   Type.—Cuttings.
   Blooming period.—April to November.
   Time to initiate roots.—Approximately 16 days.
   Time to produce a rooted young plant.—Approximately 18 days.
   Root description.—Fine, white.
Branches:
   Branching habit.—Open, branching from base and stems.
   Number of later branches.—6 to 8.
Stem:
   Diameter.—4.0 to 5.0 cm.
   Length.—20 to 35 cm.
   Shape.—Cylindrical.
   Anthocyanin.—Present.
   Anthocyanin color.—RHS 185 B.
   Color of stem.—RHS 138 A.
   Length of internode.—0.5 to 0.7 cm.
   Pubescence.—Slight.
Leaves:
   Arrangement.—Alternate.
   Shape.—Elongated oval.
   Attachment.—Sessile.
   Margin.—Entire.
   Apex.—Acute.
   Base.—Attenuate.
   Length.—1.5 to 4.5 cm.
   Width.—0.5 to 1.2 cm.
   Color.—Immature leaves: Upper surface: RHS 138 A Lower surface: RHS 138 B Mature leaves: Upper surface: RHS N 137 A Lower surface: RHS 137 C.
   Texture.—Upper surface: Smooth Lower surface: Smooth.
   Pubescence.—Moderate Upper surface: Sparse Lower surface: Absent.
   Venation pattern.—Pinnate.
   Venation color.—Upper surface: RHS 138 B Lower surface: RHS 137 C to RHS 137 D.
Inflorescence:
   Type.—Spike.
   Shape.—Explanate.
   Height or depth.—8.0 cm.
   Width.—4.0 cm.
Flowers:
   Peduncle.—Length: 13.0 cm Diameter: 2.0 mm Strength: Small Texture: Smooth Angle: Upright Color: RHS 138 D.
   Pedicel.—Length: 8.0 mm Diameter: 2.0 mm Color: RHS 138 D Surface texture: Smooth.
   Bud.—Diameter: 2.0 mm Length: 8.0 mm Shape: Linear to narrow oval Texture: Slightly pubescent.
   Sepals.—Number: 2 Length: 14.0 mm Width: 3.0 mm Shape: Linear Apex: Acute Base: Alternate Margin: Entire.
   Petals.—Number: 4 Shape: Ovate Margin: Entire Apex: Rounded Base: Connate Length: 15.0 mm Width: 10.0 mm Color: Upper surface: RHS NN 155 D Lower surface: RHS NN 155 D Texture (both upper and lower surfaces): Smooth.
   Number of flowers per inflorescence.—5 to 12.
   Lastingness of flowers on the plant.—3 to 4 days.
   Fragrance.—None observed.
Reproductive organs:
   Stamens.—Number per flower: 8 Length: 0.8 cm.
   Anthers.—Shape: Ovate Length: 3.0 mm Diameter: 0.5 mm Quantity: Moderate.
   Pistils.—Number: 1 Length: 0.15 cm Shape: Four-lobed.
   Style.—Shape: Linear
Fruit and seed set: None observed.

### COMPARISON WITH COMMERCIAL VARIETIES

The new Gaura variety ‘KLEGL10716’ differs from the commercial comparison variety ‘Crimson Butterflies’ (U.S. Plant Pat. No. 13,189), in that ‘Crimson Butterflies’ has crimson leaves, while ‘KLEGL10716’ has green leaves. In addition, ‘Crimson Butterflies’ has a very compact plant habit, while ‘KLEGL10716’ has an upright, bushy habit.

We claim:

1. A new and distinct variety of Gaura plant named ‘KLEGL10716’ as shown and described herein.
